(19) United States
(12) Patent Application Publication
(10) Pub. No.: US 2021/0393894 A1
(43) Pub. Date: Dec. 23, 2021

(54) DOWNSTREAM DELIVERY OF AGENTS IN SOLID ORGAN TRANSPLANTATION

(71) Applicant: THE REGENTS OF THE UNIVERSITY OF CALIFORNIA, Oakland, CA (US)

(72) Inventor: Abbas ARDEHALI, Los Angeles, CA (US)

(21) Appl. No.: 16/963,450

(22) PCT Filed: Jan. 23, 2019
(86) PCT No.: PCT/US19/14671

§ 371 Date: Jul. 20, 2020

Related U.S. Application Data
(60) Provisional application No. 62/620,525, filed on Jan. 23, 2018.

(51) Int. Cl. A61M 15/00; A61M 5/14

(52) U.S. Cl. CPC A61M 15/009; A61M 5/14

(57) ABSTRACT

The present invention provides systems and methods for downstream delivery of agents. The invention includes membranes that are attachable to the inner wall of a blood vessel and deposits one or more agents into a subject's bloodstream. The invention is able to treat locations downstream from the membrane attachment site. In certain embodiments, the invention is useful in solid organ transplantation, where at the time of transplanting the organ, the membrane is attached to an artery feeding into the transplanted organ and delivers agents such as immunosuppressives and gene therapy to achieve a high level of immunosuppressive medication within the transplanted solid organ while minimizing the systemic side-effects. The invention enhances solid organ function and minimizes adverse effects, leading to a better clinical outcome for the recipients. The system and methods can also be applied to autologous vascularized composite grafts.

### CROSS-REFERENCE TO RELATED APPLICATIONS

This application claims priority to U.S. Provisional Patent Application No. 62/620,525, filed Jan. 23, 2018, the contents of which are incorporated by reference herein in its entirety.

### BACKGROUND OF THE INVENTION

Solid organ transplantation has evolved to become an effective therapy for subjects with end-stage organ diseases. An integral component of solid organ transplant success has been the administration of effective immunosuppressant agents to the recipient subjects. These medications suppress the recipient immune system to minimize the attack of the immune system directed against the transplanted organ. The immunosuppressant medications include calcineurin inhibitors, anti-metabolites, and steroids, and many newer classes of agents.

Despite significant improvements in the types of immunosuppressive agents and the administration strategies, these medications are given systemically. As such, they have adverse effects on other organs of the recipients, such as nephrotoxicity, hepatotoxicity, and adverse effects on central nervous system and skin, and the like. Many organ transplant recipients on long term immunosuppressive medications develop remote (extra-transplanted organ) end organ dysfunction due to the adverse effects of the long term immunosuppressive medications on the remote organ.

Therefore, transplant specialists have to balance the desired effect of the immunosuppressive agents on the solid organ/recipient immune system versus the adverse effects of the immunosuppressive agents on the recipient's other organs. It is generally desired to have a higher level of these immunosuppressive medications within the transplanted solid organs and negligible levels outside of the solid organ. Lower systemic level may be required to alter the recipient's immune system, outside of the transplanted organ. By obtaining a high level of the immunosuppressive medication in the organ, one can improve the donor organ “acceptance” and longer function of the transplanted organ. By obtaining a lower systemic level of the immunosuppressive medication, one can minimize the adverse systemic effects.

Therefore, there is a need in the art for improved methods of delivering immunosuppressives and other agents to a transplanted organ or tissue. The present invention satisfies this unmet need.

### SUMMARY OF THE INVENTION

The present invention provides a method of administering downstream delivery of agents in a surgical procedure, comprising the steps of: surgically accessing an inner surface of at least one fluid passageway upstream from an organ designated for transplantation; selecting one or more agent delivery membranes or platforms comprising a therapeutic agent; attaching the one or more agent delivery membranes or platforms to the inner surface of the at least one fluid passageway; and transplanting the organ downstream from the one or more agent delivery membranes or platforms.

In one embodiment, the at least one fluid passageway includes an artery, a vein, an airway, or a gastrointestinal tract of a subject receiving the transplanted organ. In one embodiment, the at least one fluid passageway is attached to the organ designated for transplantation. In one embodiment, the method further comprises a step of perfusing the organ with a preserving solution prior to the step of transplanting the organ. In one embodiment, the organ is selected from the group consisting of: a kidney, a liver, a liver lobe, a heart, a lung, a double lung block, a heart lung block, a pancreas, a spleen, an appendage, a vascularized tissue graft, and/or any vascularized organ that may require a vascular connection. In one embodiment, the vascularized tissue graft is an autologous composite vascularized tissue graft.

In one embodiment, the agent delivery membrane or platform is in the shape of a substantially planar patch. In one embodiment, the agent delivery membrane or platform comprises a monolithic combination of a polymer carrier agent and a therapeutic agent. In one embodiment, the agent delivery membrane or platform is in the shape of a ring or a tube spanning the inner circumference of the at least one fluid passageway. In one embodiment, the agent delivery membrane or platform is attached using sutures, adhesives, or hooks. In one embodiment, an impermeable layer of material is positioned between the agent delivery membrane or platform and the inner surface of the at least one fluid passageway.

In one embodiment, the agent delivery membrane or platform delivers the therapeutic agent into the fluid passageway. In one embodiment, the therapeutic agent is delivered in the form of an aerosol into distal airways and alveoli. In one embodiment, the therapeutic agent is selected from the group consisting of: a calcineurin inhibitor, an antiproliferative agent, an mTOR inhibitor, a steroid, a thrombolytic agent, a nucleic acid, and combinations thereof. In one embodiment, the therapeutic agent is embedded within the agent delivery membrane or platform. In one embodiment, the therapeutic agent is contained in a reservoir embedded within the agent delivery membrane or platform. In one embodiment, the therapeutic agent is contained in a reservoir located external to the at least one fluid passageway and fluidly connected to the fluid passageway by one or more lumens having an opening in the agent delivery membrane or platform. In one embodiment, the therapeutic agent is delivered over a period of one or more minutes, one or more hours, one or more days, one or more months, or one or more years.

In another aspect, the present invention provides a system for administering downstream delivery of agents in an organ designated for transplantation, comprising: one or more agent delivery membranes or platforms comprising a therapeutic agent; a perfusion machine; and an organ preserving solution.

In another aspect, the present invention provides a method of administering downstream delivery of agents in a surgical procedure, comprising the steps of: surgically accessing the inner surface of at least one lumen upstream from a tissue designated for transplantation; selecting one or more agent delivery membranes or platforms comprising a therapeutic agent; attaching the one or more agent delivery membranes or platforms to the inner surface of the lumen; and transplanting the tissue downstream from the one or more agent delivery membranes or platform.

In one embodiment, the lumen supports a liquid fluid stream, a semi-solid fluid stream, a gaseous fluid stream, or combinations thereof. In one embodiment, the lumen is a bile duct, a lymph duct, the intestines, a ureter, the bladder, the urethra, the trachea, a bronchus, and the esophagus.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1A through FIG. 1C depict exemplary membranes/platforms attached to the inner wall of a blood vessel. FIG. 1A depicts an exemplary patch-shaped membrane/platform. FIG. 1B depicts an exemplary ring/tube-shaped membrane/platform. FIG. 1C depicts a cross-sectional view of an exemplary membrane/platform attached to the vessel wall.

FIG. 2 is a flowchart for an exemplary method of providing downstream delivery of agents to a transplanted organ.

FIG. 3 depicts an isolated organ having an exemplary agent delivery membrane/platform attached to the inner wall of an artery.

### DETAILED DESCRIPTION

The present invention provides systems and methods for downstream delivery of agents. The invention includes membranes/platforms that are attachable to the inner wall of a blood vessel and deposits one or more agents into a subject's bloodstream. The invention is able to treat locations downstream from the membrane/platform attachment site. In certain embodiments, the invention is useful in solid organ transplantation, where at the time of transplanting the organ, the membrane/platform is attached to an artery feeding into the transplanted organ and delivers agents such as immunosuppressives and gene therapy to achieve a high level of immunosuppressive medication within the transplanted solid organ while minimizing the systemic side-effects. The invention enhances solid organ function and minimizes adverse effects, leading to a better clinical outcome for the recipients. The system and methods can also be applied to autologous vascularized composite grafts.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of for example ±20%, ±10%, ±5%, ±1%, or ±0.1% from the specified value, as such variations are appropriate to perform the disclosed methods.

As used here, “biocompatible” refers to any material, which, when implanted in a mammal, does not provoke an adverse response in the mammal. A biocompatible material, when introduced into an individual, is not toxic or injurious to that individual, nor does it induce immunological rejection of the material in the mammal.

The term “biodegradable” includes polymers, compositions and formulations, such as those described herein, that are intended to degrade during use. Biodegradable polymers typically differ from non-biodegradable polymers in that the former may be degraded during use. In one embodiment, such use involves in vivo use, such as in vivo therapy. In another embodiment, such use involves in vitro use. In general, biodegradation involves the degradation of a biodegradable polymer into its component subunits, or digestion, e.g., by a biochemical process, of the polymer into smaller, non-polymeric subunits. Two types of biodegradation may generally be identified. For example, biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. Further, biodegradation may involve cleavage of a bond (whether covalent or otherwise) internal to side chain or that connects a side chain to the polymer backbone. For example, a therapeutic agent or other chemical moiety attached as a side chain to the polymer backbone may be released by biodegradation. In one embodiment, at least one type of biodegradation may occur during use of a polymer. As used herein, the term “biodegradation” encompasses all known types of biodegradation.

As used herein, the term “bioresorbable” encompasses material that can be broken down by either chemical or physical processes upon interaction with a physiological environment. The bioresorbable material can erode or dissolve. A bioresorbable material serves a temporary function in the body, such as supporting a lumen or drug delivery, and is then degraded or broken into components that are metabolizable or excretable over a period of time from minutes to years while maintaining any requisite structural integrity in that same time period.

As used herein, the terms “biocompatible polymer” and “biocompatibility” when used in relation to polymers are recognized in the art. For example, biocompatible polymers include polymers that are generally neither toxic to the host, nor degrade (if the polymer degrades) at a rate that produces monomeric or oligomeric subunits or other byproducts at toxic concentrations in the host. In one embodiment, biodegradation generally involves degradation of the polymer in a host, e.g., into its monomeric subunits, which may be known to be effectively non-toxic. Intermediate oligomeric products resulting from such degradation may have different toxicological properties, however, or biodegradation may involve oxidation or other biochemical reactions that generate molecules other than monomeric subunits of the polymer. Consequently, in one embodiment, toxicology of a biodegradable polymer intended for in vivo use, such as implantation or injection into a subject, may be determined after one or more toxicity analyses. It is not necessary that any subject composition have a purity of 100% to be deemed biocompatible; indeed, it is only necessary that the subject compositions be biocompatible as set forth above. Hence, a subject composition may comprise polymers comprising 99%, 98%, 97%, 96%, 95%, 90%, 85%, 80%, 75% or even less of biocompatible polymers, e.g., including polymers and other materials and excipients described herein, and still be biocompatible.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

As used herein, the term “polymer” refers to a molecule composed of repeating structural units typically connected by covalent chemical bonds. The term “polymer” is also meant to include the terms copolymer and oligomers. Polymers encompass degradeable and nondegradable formulations.

As used herein, the term “patient,” “subject,” “individual,” and the like are used interchangeably herein, and refer to any animal, or cells thereof, whether in vitro or in situ, amenable to the methods described herein. In certain non-limiting embodiments, the patient, subject or individual is a mammal, non-limiting examples of which include a primate, dog, cat, goat, horse, pig, mouse, rat, rabbit, and the like, that is in need of bone formation. In some embodiments of the present invention, the subject is a human being. In such embodiments, the subject is often referred to as an “individual” or a “patient.” The terms “individual” and “patient” do not denote any particular age

A “therapeutic” treatment is a treatment administered to a subject who exhibits signs of pathology, for the purpose of diminishing or eliminating those signs.

The phrase “therapeutically effective amount,” as used herein, refers to an amount that is sufficient or at least partially effective to prevent or treat (delay or prevent the onset of, prevent the progression of, inhibit, decrease or reverse) a disease or condition, including alleviating symptoms of such diseases.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The present invention includes a platform, such as a membrane, that can be used as a drug delivery system comprising one or more therapeutic agents. In one embodiment, the invention includes a drug delivery system that facilitates a controlled release of one or more therapeutic agents to a subject's bloodstream, directed downstream towards a transplanted organ or tissue. The present invention further includes methods of surgically attaching the membrane platform to the inner surface of a subject's blood vessel to deliver one or more therapeutic agents to a downstream site in a subject's body, such as a transplanted organ or tissue. In various embodiments, the membrane platform can be attached to the inner surface of any lumen in a subject's body to deliver one or more therapeutic agents into a fluid stream passing through the lumen.

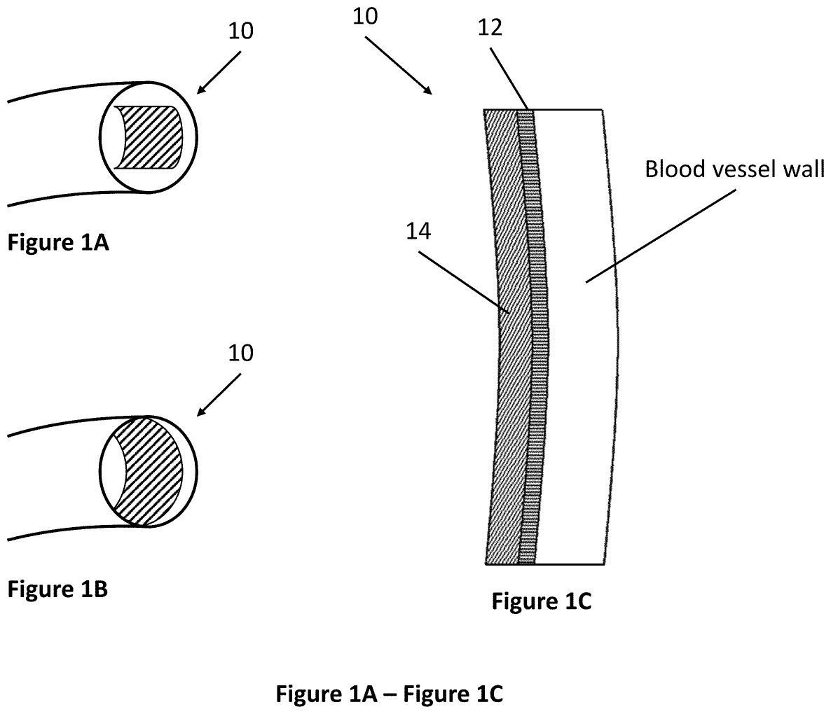

Referring now to FIG. 1A through FIG. 1C, an exemplary platform represented by membrane 10 is depicted attached to the inner surface of a blood vessel. Membrane 10 is a substantially planar material that can be attached to a blood vessel in any suitable manner, such as by sutures, adhesives, or hooks. Membrane 10 can have any suitable shape, such as a patch (FIG. 1A) or a ring/tube that spans the entire circumference of the blood vessel (FIG. 1B). Membrane 10 can have one or more layers. The one or more layers can deliver one or more agents into the bloodstream passing through the blood vessel at the attachment site. In some embodiments, membrane 10 comprises a base layer 12 positioned between a delivery layer 14 and a blood vessel wall (FIG. 1C). Base layer 12 can include an adhesive to secure delivery layer 14 to the blood vessel wall. In some embodiments, base layer 12 can be impermeable, such that agents contained in delivery layer 14 do not leak into the blood vessel wall. In some embodiments, base layer 12 can be radiopaque, such that the location of membrane 10 may be ascertained via x-ray or other imaging means. In some embodiments, membrane 10 can include one or more degradative layers on top of a delivery layer 14, such that the delivery of agents is delayed until the one or more degradative layers separate from membrane 10.

Membrane 10 can include one or more suitable agents for delivery to a subject's bloodstream. In some embodiments, the agent is an immunosuppressive. Suitable immunosuppressives encompass any drug or therapeutic that lowers a subject's ability to reject a transplanted organ or tissue, including but not limited to: calcineurin inhibitors (tacrolimus, cyclosporine), antiproliferative agents (mycophenolate mofetil, mycophenolate sodium, azathioprine), mTOR inhibitors (sirolimus), and steroids (prednisone). However, the invention should not be limited to the delivery of immunosuppressives. Rather, delivery of any therapeutic agent is included in the invention. For example, the membrane platform of the invention can be used to deliver one or more thrombolytic agents, such as urokinase, anistreplase, alteplase, reteplase, streptokinase, staphylokinase, and tenecteplase. It should be understood that any and all therapeutic or modulatory agents or compounds that may benefit or impact the transplanted organ or tissue are contemplated and may be used in membrane 10.

In some embodiments, the one or more agents can include nucleic acids. Nucleic acids can be used in gene therapy to replace genes in a mammal. The nucleic acids can be introduced into cells of a tissue to yield a phenotypic change in the tissue. In some embodiments, the cells of the tissue can be modified to express proteins and markers that more closely resemble those of the subject, thereby reducing or eliminating transplant rejection symptoms. A gene construct may be provided as an expression vector that includes the coding sequence for a heterologous protein operably linked to essential regulatory sequences such that when the vector is transfected into a cell, the coding sequence will be expressed by the cell. The coding sequence is operably linked to the regulatory elements necessary for expression of that sequence in the cells. The nucleotide sequence that encodes the protein may be cDNA, genomic DNA, synthesized DNA or a hybrid thereof or an RNA molecule such as mRNA. The nucleic acids can be encapsulated to enhance downstream delivery, such as in a nanospheres, liposomes, virus-like particles, and the like.

The gene construct may include a nucleotide sequence encoding a beneficial protein operably linked to the regulatory elements and may remain present in the cell as a functioning cytoplasmic molecule, a functioning episomal molecule or it may integrate into the cell's chromosomal DNA. Exogenous genetic material may be introduced into cells where it remains as separate genetic material in the form of a plasmid. Alternatively, linear DNA which can integrate into the chromosome may be introduced into a cell. When introducing DNA into a cell, reagents which promote DNA integration into chromosomes may be added. DNA sequences which are useful to promote integration may also be included in the DNA molecule. Alternatively, RNA may be introduced into the cell.

In some embodiments, membrane 10 deposits the one or more agents into the bloodstream by degradative delivery. For example, the one or more delivery layers 14 may dissolve or elute over time, thereby releasing the drug held within the delivery layers 14. In other embodiments, membrane 10 deposits the one or more agents into the bloodstream by non-degradative delivery. Non-degradative delivery may employ one or more reservoirs filled with agents for release over time. In some embodiments, reservoirs may be embedded in the one or more delivery layers 14. In other embodiments, the reservoirs may be external to the blood vessel, wherein the agents may be delivered to the bloodstream by way of one or more lumens penetrating the blood vessel wall and secured in position by membrane 10 (not pictured). In certain embodiments, the lumens terminate in the bloodstream with a one-way valve. External reservoirs may be located within the body to minimize the risk of accidental detachment. External reservoirs may also be located outside the body to facilitate replenishment of agents or the exchange of reservoirs. Agent delivery from reservoirs may be performed passively or actively using a pump. In various embodiments, delivery of the one or more agents can occur continuously or intermittently over one or more minutes, one or more hours, one or more days, one or more weeks, one or more months, or one or more years.

Membrane 10 can be constructed from any suitable material, including durable polymers, bioresorbable polymers, biocompatible polymers, and any other material suitable for the delivery of therapeutic agents. The polymers can be categorized as biodegradable and non-biodegradable. Biodegradable polymers degrade in vivo as a function of chemical composition, method of manufacture, and implant structure. Synthetic and natural polymers or both can be used. Examples of synthetic polymers include polyanhydrides, polyhydroxyacids such as polylactic acid, polyglycolic acids and copolymers thereof, polyesters, polyamides, polyorthoesters, and some polyphosphazenes. Examples of naturally occurring polymers include proteins and polysaccharides such as collagen, hyaluronic acid, albumin and gelatin. The one or more agents can be encapsulated within, throughout, and/or on the surface of the polymers.

In some embodiments, the synthetic polymer or copolymer is prepared from at least one of the group of monomers consisting of acrylic acid, methacrylic acid, ethyleneimine, crotonic acid, acrylamide, ethyl acrylate, methyl methacrylate, 2-hydroxyethyl methacrylate, lactic acid, glycolic acid, .ε-caprolactone, acrolein, cyanoacrylate, bisphenol A, epichlorhydrin, hydroxyalkylacrylates, siloxane, dimethylsiloxane, ethylene oxide, ethylene glycol, hydroxyalkyl-methacrylates, N-substituted acrylamides, N-substituted methacrylamides, N-vinyl-2-pyrrolidone, 2,4-pentadiene-1-ol, vinyl acetate, acrylonitrile, styrene, p-amino-styrene, p-amino-benzyl-styrene, sodium styrene sulfonate, sodium 2-sulfoxyethyl methacrylate, vinyl pyridine, aminoethyl methacrylates, 2-methacryloyloxy-trimethylammonium chloride, N,N′-methylenebisacrylamide-, ethylene glycol dimethacrylates, 2,2′-(p-phenylenedioxy)-diethyl dimethacrylate, divinylbenzene, and triallylamine, methylenebis-(4-phenyl-isocyanate).

A variety of polymers from synthetic and/or natural sources can be used to produce the polymer matrix of the membranes/platforms of the invention. For example, lactic or polylactic acid or glycolic or polyglycolic acid can be utilized to form poly(lactide) (PLA) or poly(L-lactide) (PLLA) nanofibers or poly(glycolide) (PGA) nanofibers. The polymer matrix can also be made from more than one monomer or subunit thus forming a co-polymer, terpolymer, etc. For example, lactic or polylactic acid and be combined with glycolic acid or polyglycolic acid to form the copolymer poly(lactide-co-glycolide) (PLGA). Other copolymers of use in the invention include poly(ethylene-co-vinyl) alcohol). In an exemplary embodiment, the polymer matrix can comprise a polymer or subunit which is a member selected from an aliphatic polyester, a polyalkylene oxide, polydimethylsiloxane, polyvinylalcohol, polylysine, and combinations thereof. In another exemplary embodiment, the polymer matrix can comprises two different polymers or subunits which are members selected from an aliphatic polyester, a polyalkylene oxide, polydimethylsiloxane, polyvinylalcohol, polylysine, and combinations thereof. In another exemplary embodiment, the polymer matrix comprises three different polymers or subunits which are members selected from an aliphatic polyester, a polyalkylene oxide, polydimethylsiloxane, polyvinylalcohol, polylysine, and combinations thereof. In an exemplary embodiment, the aliphatic polyester is linear or branched. In another exemplary embodiment, the linear aliphatic polyester is a member selected from lactic acid (D- or L-), lactide, poly(lactic acid), poly(lactide) glycolic acid, poly(glycolic acid), poly(glycolide), glycolide, poly(lactide-co-glycolide), poly(lactic acid-co-glycolic acid), polycaprolactone and combinations thereof. In another exemplary embodiment, the aliphatic polyester is branched and comprises at least one member selected from lactic acid (D- or L-), lactide, poly(lactic acid), poly(lactide) glycolic acid, poly(glycolic acid), poly(glycolide), glycolide, poly(lactide-co-glycolide), poly(lactic acid-co-glycolic acid), polycaprolactone and combinations thereof which is conjugated to a linker or a biomolecule.

As another example, the polymer may be formed from functionalized polyester graft copolymers. The functionalized graft copolymers are copolymers of polyesters, such as poly(glycolic acid) or poly(lactic acid), and another polymer including functionalizable or ionizable groups, such as a poly(amino acid). In another embodiment, polyesters may be polymers of α-hydroxy acids such as lactic acid, glycolic acid, hydroxybutyric acid and valeric acid, or derivatives or combinations thereof. The inclusion of ionizable side chains, such as polylysine, in the polymer has been found to enable the formation of more highly porous particles, using techniques for making microparticles known in the art, such as solvent evaporation. Other ionizable groups, such as amino or carboxyl groups, may be incorporated, covalently or noncovalently, into the polymer to enhance porosity. For example, polyaniline could be incorporated into the polymer. These groups can be modified further to contain hydrophobic groups capable of binding load molecules.

In some embodiments, the polymer can include one or more of the following: polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, poly-ε-caprolactone, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly(ethylene terephthalate), poly(vinyl acetate), polyvinyl chloride, polystyrene, polyvinyl pyrrolidone, pluronics, polyvinylphenol, saccharides (e.g., dextran, amylose, hyalouronic acid, poly(sialic acid), heparans, heparins, etc.); poly (amino acids), e.g., poly(aspartic acid) and poly(glutamic acid); nucleic acids and copolymers thereof.

In some embodiments, the polymer can include one or more of the following: peptide, saccharide, poly(ether), poly(amine), poly(carboxylic acid), poly(alkylene glycol), such as poly(ethylene glycol) (“PEG”), poly(propylene glycol) (“PPG”), copolymers of ethylene glycol and propylene glycol and the like, poly(oxyethylated polyol), poly(olefinic alcohol), poly(vinylpyrrolidone), poly(hydroxypropylmethacrylamide), poly(α-hydroxy acid), poly(vinyl alcohol), polyphosphazene, polyoxazoline, poly(N-acryloylmorpholine), polysialic acid, polyglutamate, polyaspartate, polylysine, polyethyeleneimine, biodegradable polymers (e.g., polylactide, polyglyceride and copolymers thereof), polyacrylic acid.

In some embodiments, membrane 10 can be provided with a selection of tools useful for organ or tissue transplants to form a system for administering downstream delivery of agents in an organ or tissue designated for transplantation. For example, isolated organs may be connected to a perfusion machine to perfuse the organ with an organ preserving solution. One or more membranes 10 may be provided with the perfusion machine and the organ preserving solution, such that the isolated organ may be fitted with a membrane 10 prior to connecting the organ to the perfusion machine. In this manner, one or more agents may be deposited into the circulating preserving solution and delivered to the organ by the perfusion machine, conditioning the organ before it reaches a subject.

The present invention also provides methods for using membranes/platforms to deliver one or more agents to a downstream site in need thereof. The methods surgically attach the membranes/platforms to the inner surface of a blood vessel to deposit one or more agents into the bloodstream. In some embodiments, the methods include the process of surgically attaching the membranes/platforms to a blood vessel upstream from a transplanted organ or tissue. In some embodiments, the membrane or platform may release micro-spheres or nano-spheres that may modulate the function of a downstream organ. The positioning of the membranes/platforms thereby enables the administration of agents into the bloodstream to be carried downstream to a transplanted organ or tissue, which may result in better acceptance of the donor organ or tissue by reducing the chance of rejection. A higher, localized concentration of drugs such as immunosuppressives medications in the transplanted organ or tissue may allow for lower systemic concentration of the medications and a reduction of related side-effects.

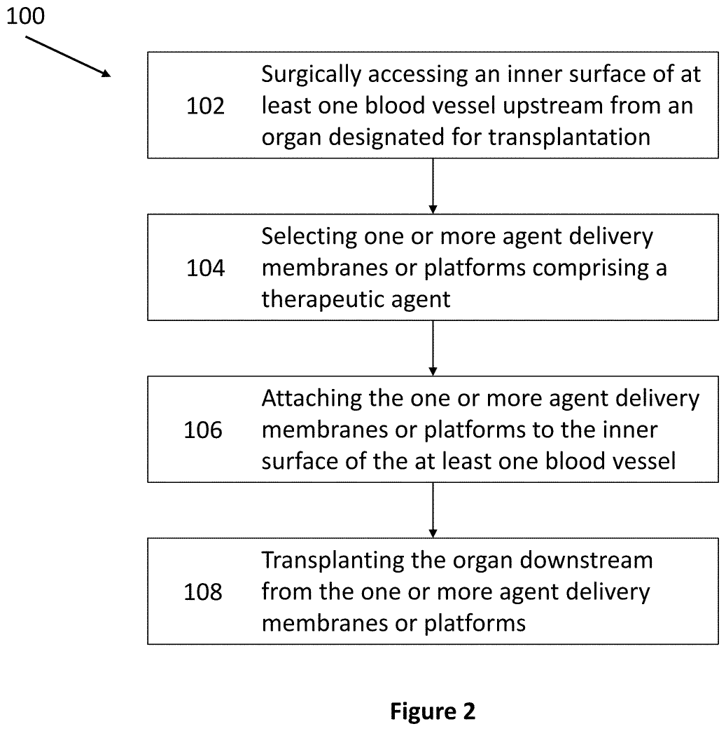

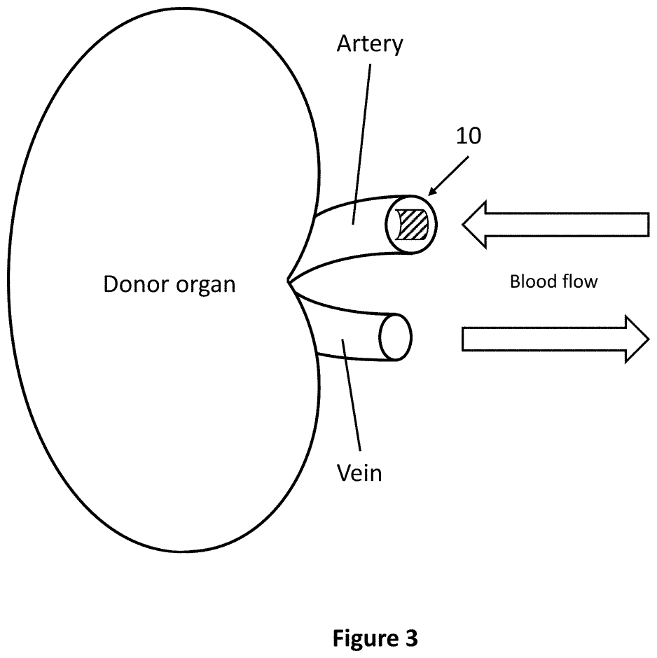

Referring now to FIG. 2, an exemplary method 100 of administering downstream delivery of agents in a surgical procedure is depicted. Method 100 begins with step 102 of surgically accessing an inner surface of at least one blood vessel upstream from an organ designated for transplantation. In some embodiments, the at least one blood vessel includes the artery (FIG. 3) or vein of the isolated organ. In other embodiments, the at least one blood vessel includes the arteries or veins of the subject receiving the organ. In step 104, one or more agent delivery membranes/platforms comprising a therapeutic agent of the present invention are provided and selected for implantation, such as the patch membrane or the ring/tube membrane described elsewhere herein. In step 106, the one or more agent delivery membranes/platforms are attached to the inner membrane of the at least one blood vessel. In step 108, an organ is transplanted downstream from the one or more agent delivery membranes/platforms.

In some embodiments, the agent delivery membranes/platforms may be attached to the arteries of the subject receiving the organ transplant prior to suturing the blood vessels of the organ to the blood vessels of the subject. Agents deposited into the bloodstream of the arteries would advantageously flow towards tissues downstream from the arteries, minimizing exposure of the agents to tissues that do not need treatment.

In some embodiments, the agent delivery membranes/platforms may be attached to the artery or vein of the isolated organ designated for transplantation. The isolated organ can be any suitable organ or tissue, including a kidney, a liver, a liver lobe, a heart, a lung, a double lung block, a heart lung block, a pancreas, a spleen, and/or any vascularized organ that may require a vascular connection. For example, in some embodiments, the agent delivery membranes/platforms may be attached to a portal vein of an isolated liver.

In some embodiments, the agent delivery membranes/platforms may be attached to an artery or vein of a vascularized tissue graft. The tissue can be an autologous tissue graft or an allogeneic tissue graft. In some embodiments, the vascularized tissue is an appendage, such as a hand, a foot, an arm, and a leg. In some embodiments, the graft tissue is a segment of an artery or a vein. In some embodiments, the vascularized tissue is composite vascularized tissue, such as a free flap or a pedicled flap used in reconstruction surgery.

In some embodiments, the agent delivery membranes/platforms may be attached to the artery or vein of the organ at the moment of extraction, such that conditioning of the isolated organ may commence prior to transplantation. For example, the isolated organ with an attached agent delivery membrane/platform may be connected to a perfusion machine that perfuses the organ with a preserving solution, whereupon the agents from the delivery membrane/platform may be deposited into the preserving solution and carried into the organ. The agent delivery membranes/platforms may remain attached when the organ is transplanted into a subject. In other embodiments, the agent delivery membranes/platforms may be attached to the artery or vein of the organ during the organ transplant procedure, just prior to suturing the blood vessels of the isolated organ to the blood vessels of the subject receiving the organ. Agents deposited into the bloodstream of the donor organ artery would advantageously flow downstream directly into the organ, minimizing exposure of the agents to the rest of the body.

In some embodiments, the methods relate to using the membranes/platforms of the present invention in any suitable fluid passageway or lumen to administer downstream delivery of agents. The membranes/platforms can be attached to an inner surface of the fluid passageway or lumen and can deposit one or more agents into a fluid stream passing through the lumen. In some embodiments, the membrane/platforms can be attached to a lumen supporting a liquid or a semi-solid fluid stream, such as a bile duct, a lymph duct, the intestines, a ureter, the bladder, and the urethra. In some embodiments, the membrane/platform can be attached to a lumen supporting a gaseous fluid stream, such as the trachea and bronchi of the lungs. In some embodiments, the membrane/platform can be attached to a lumen supporting a liquid, a semi-solid, and a gaseous fluid stream, such as the esophagus. In various embodiments, the delivery of therapeutic agents into a fluid passageway or lumen may be selected depending upon the type of stream within the passageway or lumen. For example, the therapeutic agent can be delivered in the form of a solid, a liquid, a gel, an aerosol, a vapor, a powder, a microparticle, a nanoparticle, an encapsulated particle (such as a microsphere or a nanosphere), and combinations thereof into a liquid, solid, semi-solid, or gaseous fluid stream.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of administering downstream delivery of agents in a surgical procedure, comprising the steps of:
surgically accessing an inner surface of at least one fluid passageway upstream from an organ designated for transplantation;
selecting one or more agent delivery membranes or platforms comprising a therapeutic agent;
attaching the one or more agent delivery membranes or platforms to the inner surface of the at least one fluid passageway; and
transplanting the organ downstream from the one or more agent delivery membranes or platforms.

2. The method of claim 1, wherein the at least one fluid passageway includes an artery, a vein, an airway, or a gastrointestinal tract of a subject receiving the transplanted organ.

3. The method of claim 1, wherein the at least one fluid passageway is attached to the organ designated for transplantation.

4. The method of claim 3, further comprising a step of perfusing the organ with a preserving solution prior to the step of transplanting the organ.

5. The method of claim 1, wherein the organ is selected from the group consisting of: a kidney, a liver, a liver lobe, a heart, a lung, a double lung block, a heart lung block, a pancreas, a spleen, an appendage, a vascularized tissue graft, and/or any vascularized organ that may require a vascular connection.

6. The method of claim 5, wherein the vascularized tissue graft is an autologous composite vascularized tissue graft.

7. The method of claim 1, wherein the agent delivery membrane or platform is in the shape of a substantially planar patch.

8. The method of claim 1, wherein the agent delivery membrane or platform comprises a monolithic combination of a polymer carrier agent and a therapeutic agent.

9. The method of claim 1, wherein the agent delivery membrane or platform is in the shape of a ring or tube spanning the inner circumference of the at least one fluid passageway.

10. The method of claim 1, wherein the agent delivery membrane or platform is attached using sutures, adhesives, or hooks.

11. The method of claim 1, wherein an impermeable layer of material is positioned between the agent delivery membrane or platform and the inner surface of the at least one fluid passageway.

12. The method of claim 1, wherein the agent delivery membrane or platform delivers the therapeutic agent into the fluid passageway.

13. The method of claim 12, wherein the therapeutic agent is delivered in the form of an aerosol into distal airways and alveoli.

14. The method of claim 1, wherein the therapeutic agent is selected from the group consisting of: a calcineurin inhibitor, an antiproliferative agent, an mTOR inhibitor, a steroid, a thrombolytic agent, a nucleic acid, and combinations thereof.

15. The method of claim 1, wherein the therapeutic agent is embedded within the agent delivery membrane or platform.

16. The method of claim 1, wherein the therapeutic agent is contained in a reservoir embedded within the agent delivery membrane or platform.

17. The method of claim 1, wherein the therapeutic agent is contained in a reservoir located external to the at least one fluid passageway and fluidly connected to the fluid passageway by one or more lumens having an opening in the agent delivery membrane or platform.

18. The method of claim 11, wherein the therapeutic agent is delivered over a period of one or more minutes, one or more hours, one or more days, one or more months, or one or more years.

19. A system for administering downstream delivery of agents in an organ designated for transplantation, comprising:
one or more agent delivery membranes or platforms comprising a therapeutic agent;
a perfusion machine; and
an organ preserving solution.

20. A method of administering downstream delivery of agents in a surgical procedure, comprising the steps of:
surgically accessing an inner surface of at least one lumen upstream from a tissue designated for transplantation;
selecting one or more agent delivery membranes or platforms comprising a therapeutic agent;
attaching the one or more agent delivery membranes or platforms to the inner surface of the lumen; and
transplanting the tissue downstream from the one or more agent delivery membranes or platforms.

21. The method of claim 20, wherein the lumen supports a liquid fluid stream, a semi-solid fluid stream, a gaseous fluid stream, or combinations thereof.

22. The method of claim 20, wherein the lumen is a bile duct, a lymph duct, the intestines, a ureter, the bladder, the urethra, the trachea, a bronchus, and the esophagus.
